(19) United States
(12) Patent Application Publication
(10) Pub. No.: US 2010/0225494 A1
(43) Pub. Date: Sep. 9, 2010

(54) Fluid Flow Outage Safety and Warning Device

(76) Inventor: Gregory L. Thorpe, New Rochelle, NY (US)

Correspondence Address: Gregory L. Thorpe, 277 Mayflower Avenue, New Rochelle, NY, 10801, US

(21) Appl. No.: 12/691,432

(22) Filed: Jan. 21, 2010

Related U.S. Application Data
(60) Provisional application No. 61/147,085, filed on Jan. 24, 2009.

(51) Int. Cl. G08B 17/10; A61M 5/40

(52) U.S. Cl. 340/632; 604/127

(57) ABSTRACT

An intravenous fluid outage warning device comprising:

A hollow body having a fluid chamber provided thereof, a fluid inlet and fluid outlet in communication with said fluid chamber, said fluid inlet being made such that it is capable of allowing entry of intravenous fluid from a fluid containing member and the fluid outlet being disposed in a manner wherein the intravenous fluid can flow through towards a standard fluid drip administering means for medication or hydration, float means disposed within the fluid chamber being made such that it is floatable when fluid is contained in the fluid chamber and capable of plugging the mouth of the fluid outlet when said fluid is already depleted, thereby preventing the entry of air to the fluid drip administering means, an activating member provided on said hollow body being held thereof in a manner wherein it is capable of tilting by the float means as the level of the fluid in the fluid chamber goes down, an electric contact circuit assembly being held on said hollow body being capable of completing an electrical circuit, by said activating member when tilted by the float means, thereby activating an alarm signal at the attendant nurse station, and stopper member provided within the fluid chamber and disposed below the fluid inlet.

The fluid chamber and stopper member comprise the standard model and when such fluid chamber and stopper member are combined with the electric contact circuit assembly and activating member, the total combination of components comprises the premium model.

### TECHNICAL FIELD

The present invention relates generally to an apparatus for monitoring intravenous drips and more specifically to an intravenous fluid outage warning device. However, the device in different sizes serves the same functions for any fluid flow process when a low or empty level exists.

### BACKGROUND OF THE INVENTION

In the infusion of intravenous fluid to a patient for medication or saline for prevention of dehydration, a bag containing the fluid is utilized wherein it is attached to a filter chamber leading to a reservoir having a piece of tubing extending therefrom. The fluid from the tubing is fed into the vein of the patient through a needle provided thereof. During the process, the attending nurse usually conducts frequent monitoring of the fluid level to prevent the possibility of the air in the injection tube entering the blood vessel of the patient when the fluid supply runs dry. Failure to conduct such a routine may result in an air embolism, medical complications or endanger the life of the patient. The monitoring routine, however, consumes a lot of the nurse's time and adds pressure to the his/her usually hectic work schedule.

To solve the aforesaid problem, costly electronic devices for monitoring the rate and duration of intravenous drip have been employed for such purpose. One of these monitoring devices is the flow rate monitor and totalizing count display apparatus disclosed in U.S. Pat. No. 4,321,461. Said U.S Patent describes a flow rate monitor and totalizing count display apparatus that utilizes a pulsed infrared light source and receiver and include alarm means for signaling a no-drop condition and if a preselected count is reached. While this apparatus works effectively in its intended purpose, however its structure is too complicated, difficult and time consuming to operate.

One monitoring device that is quite simpler than the aforementioned is disclosed in Taiwan Patent No. 381494. Said patent includes a housing having a point bar whose top end is inserted into the solution bottle and its outlet communicating with a solution tube. A float being fitted inside the pointed bar being capable of automatically activating a buzzer when lowered at a certain liquid level therein through conductivity of magnets provided at the structure. The problem with this device is that the float and the magnet do not react sensitively with each other due to insufficient buoyancy and magnetic force, such that its function is not precise. The cost of such other devices has limited their use and therefore most patients are left at risk as well as such other devices require electricity to work and leaves field hospital patients at risk.

### SUMMARY OF THE INVENTION

It is therefore the primary object of the present invention to provide an intravenous fluid outage warning device that can remedy the above drawbacks of the prior art.

Another object is to provide a device wherein a float means is provided in an intravenous fluid chamber, being disposed thereof in a manner wherein it is capable of settling into the mouth of the fluid outlet when the fluid is already consumed. The disposition of the float means on the fluid outlet mouth prevents air from flowing into the patient's vein. Furthermore, the downward movement of the float means with respect to the decreasing level of the fluid in the chamber facilitates movement of a pivoting member thereby completing an electric circuit capable of activating an alerting means provided in the nurse station.

Still another object of the present invention is to provide an intravenous fluid outage warning device which is very simple in structure which operates without electricity and therefore can be manufactured at a much lower expense: thereby providing the medical field with the ability to protect all patients receiving intravenous fluids.

Other objects and advantages of the present invention can be fully appreciated upon reading the following description taken in conjunction with the accompanying drawings.

### BRIEF DESCRIPTION OF THE SEVERAL VIEWS OF THE DRAWINGS

FIG. 1 is a perspective view of the intravenous fluid outage warning device of the present invention,

FIG. 2 is a cross-sectional view taken along line 2-2 of FIG. 1,

FIG. 3 is an illustrative presentation showing the present device in its position of use,

FIG. 4 is a perspective view of another embodiment of the present invention,

FIG. 5 is a cross-sectional view taken along line 5-5 of FIG. 4, and

FIG. 6 is another cross-sectional view showing a variation of the float means of the present invention.

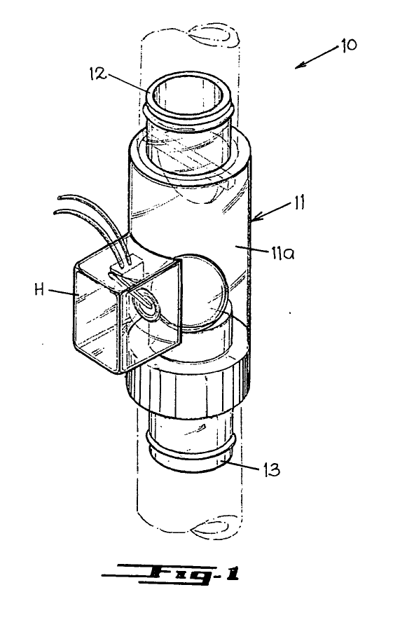

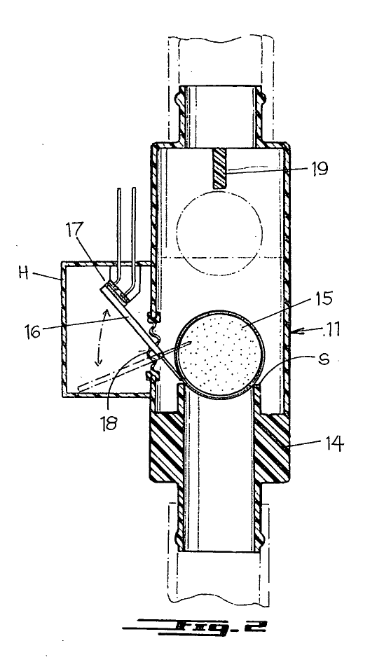

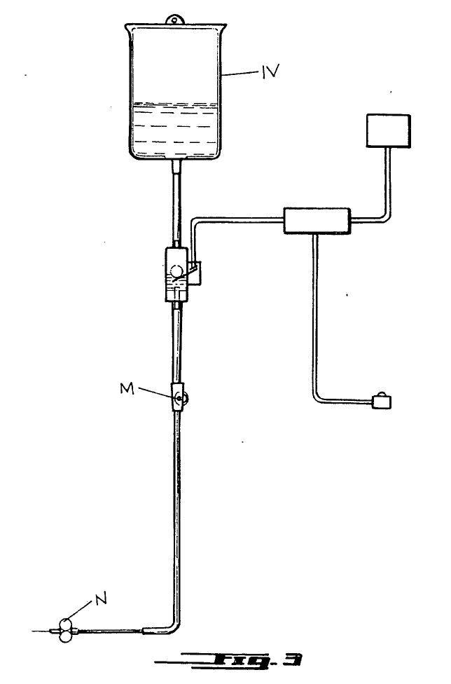

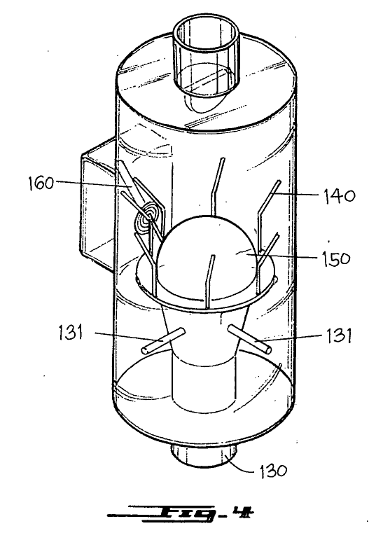

### DETAILED DESCRIPTION OF THE SEVERAL EMBODIMENTS OF THE INVENTION

Referring to the drawings, there is shown an intravenous fluid outage warning device generally designated as 10 comprising a hollow body 11 having a fluid chamber 11a in connection with a fluid inlet 12 and fluid outlet 13 provided thereof. Said fluid inlet 12 is being made such that it is capable of connecting with an intravenous fluid containing member IV, and to fluid outlet 13 which is capable of attachment to a standard drip administering means consisting of a drip rate control mechanism M and infusing means N. Said drip rate control mechanism facilitates the flow of drip fluid to the patient's vein through said infusing needle N. Hollow body 11 is preferably provided with a thickened lower portion 14 wherein the upper portion of fluid outlet 13 is passing through. Fluid outlet 13 is preferably cylindrical in shape such that the mouth communicating with the fluid chamber defines a round concave seat S. A float means 15 disposed within fluid chamber 11a being made such that it is capable of floating when there is liquid in the fluid chamber or settles into seat S and plugs the mouth of the fluid outlet once the fluid is already depleted. Float means is preferably made of light floatable material that includes styrofoam and the like. Said float means may be coated with soft foam rubber to provide an airtight seal when it fits with the mouth of the fluid outlet. The foam rubber may be coated with non stickable coating, such as teflon, to prevent the float means from sticking to the fluid outlet surface. Further provided on said hollow body 11 is a tiltable activating member 16 having its inner portion disposed within the fluid chamber and its outer portion capable of communication with an electrical assembly 17 mounted on the hollow body which in turn is capable of transmitting alarm signals to an attendant's station. Said activating member is preferably an elongated piece being held in a sealed manner in the hollow body by a flexible piece 18 such that tilting movement can be facilitated. The activating member 16 is being made such that its outer portion is heavier than the inner portion, such that it assumes an initially tilted position when fluid is contained in the fluid chamber. Such initial tilting position is formed in a manner wherein the outer portion of the activating member is spaced from the electric terminals of the electrical contacts assembly 17 and the inner portion disposed below the float means 15. The gradual depletion of fluid in the fluid chamber shall result in the float means moving down and to be in contact with the inner portion of the activating member, thus causing gradual inverse tilting of the activating member. When the fluid is totally depleted, the float means will be in a position wherein it is resting in seat S with its underside plugging the mouth of the fluid outlet and totally air-sealing it. Also in this position, the activating member is in a totally inversed tilted position wherein its outer portion is in contact with the terminals of the electrical assembly and its inner portion disposed in a downwardly directed manner and abutting the float means. In this position, the alerting means A in the attendant station is activated thereby giving signals to the nurse that the fluid in the fluid containing member is already depleted.

To prevent blocking of the fluid inlet by the float means, a stopper member 19 is provided below the fluid inlet 12 and fixedly held within the hollow body. Electrical assembly 17 is further provided with housing H to prevent dust from coming in and for safety purposes.

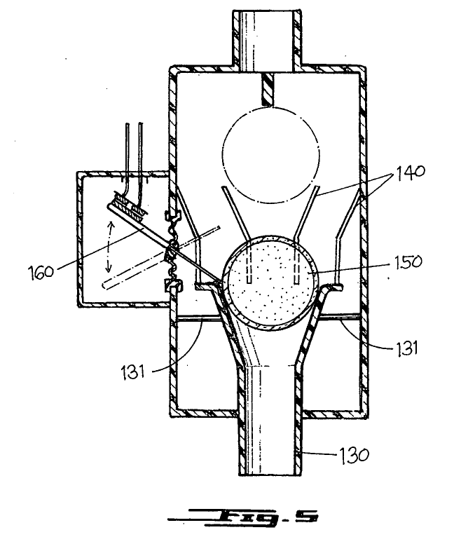

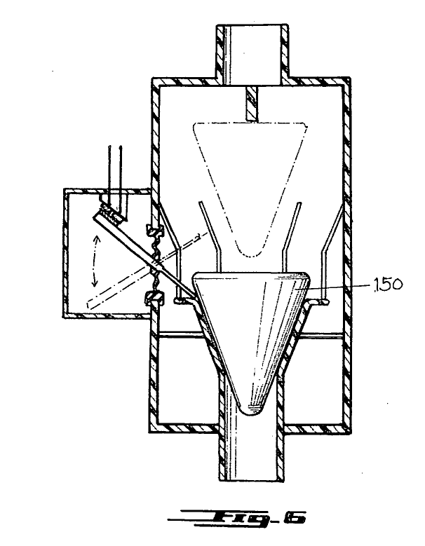

As shown in FIGS. 4 and 5 is another embodiment of the present invention wherein fluid outlet 130 is in the form of a funnel such that a wider seat area is formed. A plurality of pre-shaped guide means 140 are fixedly held within the hollow body disposed in a manner wherein their cooperative position thereof facilitates steady upward and downward movement of the activating member 160 and assures settlement of float means 150 in the fluid outlet 130. Said guide means can be in the form of pre-shaped rod members or other elongated members made of suitable material such as plastic and metal. Furthermore, in this variation, fluid outlet 130 may be held within the hollow body by stabilizing elongated members 131 such that the upper portion of said fluid outlet is disposed in a suspended manner in the fluid chamber.

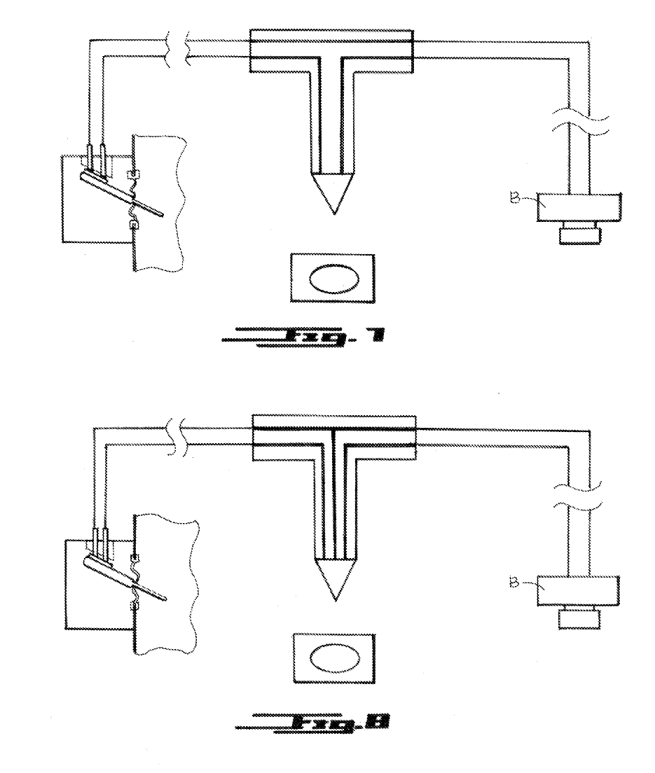

As shown in FIG. 6 is a variation of the shape of the float means 150 wherein it is made conical to conform to the shape of the funnel-like fluid outlet 130. Since a wider surface area is covered by the insertion of float means 150 in the fluid outlet 130, an airtight sealing is assured.

I claim:

1) An intravenous fluid outage warning device comprising:
A hollow body having a fluid chamber provided thereof, a fluid inlet and fluid outlet in communication with said fluid chamber, said fluid inlet being made such that it is capable of allowing entry of intravenous fluid from a fluid containing member and the fluid outlet being disposed in a manner wherein the intravenous fluid can flow therefrom towards a standard fluid drip administering means for medication or hydration, float means disposed within the fluid chamber being made such that it is floatable when fluid is contained in the fluid chamber and capable of plugging the mouth of the fluid outlet when said fluid is already depleted, thereby preventing the entry of air to the fluid drip administering means, an activating member provided on said hollow body being held thereof in a manner wherein it is capable of tilting by the float means as the level of the fluid in the fluid chamber goes down, an electric circuit assembly being held on said hollow body being capable of activation by said activating member when tilted by the float means, thereby sending alarm signal to an attendant station, and stopper member provided within the fluid chamber and disposed below the fluid inlet.

2) An intravenous fluid outage warning device according to claim 1 wherein said float means is pre-shaped to conform with the opening of the fluid outlet and made of light material that includes Styrofoam, hollow plastic, or hollow metal each covered in a soft rubber material to aid the air tight seating of the float means in the mouth of the fluid outlet.

3) An intravenous fluid outage warning device according to claim 2 wherein said float means is in a form that includes round and conical.

4) An intravenous fluid outage warning device according to claim 2 wherein said float means is being coated with soft foam rubber.

5) An intravenous fluid outage warning device according to claim 4 wherein the soaf foam rubber coating of the float means is further provided with non-stickable coating material.

6) An intravenous fluid outage warning device according to claim 5 wherein said non-stickable coating material includes teflon.

7) An intravenous fluid outage warning device according to claim 1 wherein said fluid outlet is cylindrical.

8) An intravenous fluid outage warning device according to claim 1 wherein said fluid outlet is in the form of a funnel.

9) An intravenous fluid outage warning device according to claim 1 wherein said fluid outlet is being held in a suspended manner within the hollow body.

10) An intravenous fluid outage warning device according to claim 1 wherein a plurality of pre-shaped guide means are provided in said hollow body disposed thereof in a manner wherein their cooperative positions facilitates steady tilting of the activating member and assure settlement of the float means in the fluid outlet.

11) An intravenous fluid outage warning device according to claim 7 wherein said guide means are pre-shaped rod members.

12) An intravenous fluid outage warning device according to claim 7 wherein said guide means are elongated members.
